(19) United States
(12) Patent Application Publication
(10) Pub. No.: US 2025/0356491 A1
(43) Pub. Date: Nov. 20, 2025

(54) ASSESSMENT OF MEDICAL IMAGES OF THE BRAIN

(71) Applicant: OSLO UNIVERSITETSSYKEHUS HF, Oslo (NO)

(72) Inventors: Elies FUSTER I GARCIA, Massalfassar (ES); Kyrre Eeg EMBLEM, Oslo (NO); Atle BJØRNERUD, Oslo (NO)

(21) Appl. No.: 18/729,640

(22) PCT Filed: Jan. 16, 2023
(86) PCT No.: PCT/EP2023/050885

§ 371 Date: Jul. 17, 2024

(30) Foreign Application Priority Data
Jan. 18, 2022 (GB) 2200594.6

(51) Int. Cl. G06T 7/00; G06T 7/11; G06T 7/174; G16H 30/20

(52) U.S. Cl. CPC G06T 7/0014; G06T 7/11; G06T 7/174; G16H 30/20; G06T 2207/10088; G06T 2207/20084; G06T 2207/30016; G06T 2207/30096

(57) ABSTRACT

A method of assessment of medical images of a brain is described. The method is for patients with a lesion of the brain. Lesion tissues are segmented using automated methods and a region of interest in the peri-lesion area is defined based on dilation operations. The method includes estimating the displacement field within each consecutive image pair within the stack of longitudinal medical images and then computing magnitude and divergence maps from the estimated displacement fields. This is done by computing the magnitude of the displacement field to characterise pixelwise displacement within the image field of view and divide it by the time between their associated consecutive image pair; and computing the divergence of the displacement field to characterise pixelwise compression and/or expansion phenomena within the image field of view and dividing by the time between their associated consecutive image pair.

The technical field of the present invention relates to the operation mode of switches for multi-group LEDs, which may in particular be used for or be part of an LED driver.

In general, an LED based product, e.g. a LED fixture, is driven by an LED driver. The LED fixture in general comprises multi-group LEDs, i.e. groups of LEDs which are arranged in a parallel connection. Based on the color characteristics of the multi-group LEDs applied, a color by each LED group can be generated. When the LED driver switches between the different LED groups, e.g. to generate a color pattern, only one particular LED group is powered on and the other LED groups are powered off. The switching on/off of a LED group occurs by controlling a switch, e.g. a MOSFET, which is placed in serial connection with the LED group.

Problems may arise when switching between two LED groups, wherein the forward voltage of each LED group is different. When switching from a LED group with a higher forward voltage to another LED group with a lower forward voltage, the LED driver experiences an unwanted peak-current, which may damage the switched LED group.

It is an object of the present invention to provide an improved LED driver, in particular being less prone to peak-currents, or at least to provide an alternative for known solutions.

This object is achieved by providing an LED driver configured to control a LED fixture comprising a plurality of LED groups, each LED group comprising at least one LED forming a series connection with a switch, wherein the plurality of LED groups are arranged in a parallel connection, the LED fixture further comprising a capacitor arranged in a parallel connection to the plurality of LED groups, the LED driver comprising:
a power converter for converting an input power from a power source to a current which is provided to the plurality of LED groups;
a control unit configured to control the power converter, as such the power converter provides current to plurality of LED groups, and wherein the control unit is further configured to
   control the operation mode of the switch of the LED group between a nominal mode, wherein the switch operates in a closed nominal state or an open nominal state, and
   a transition mode, wherein the switch operates in a semi-closed state, wherein the gate to source voltage of the switch of the LED group is lower than the gate to source voltage of the respective switch when operated in the nominal mode,
   switch the current from flowing through a first LED group to flowing through a second LED group, wherein the first LED group has a higher forward voltage than the second LED group, by switching the switch of the first LED group in the open nominal state and the switch of the second LED group in the semi-closed state during a transient period.

In accordance with the present invention, an LED driver is, in general, applied for powering an LED fixture. The LED fixture comprises a plurality of LED groups. Each LED group comprises at least one LED forming a series connection with a switch. The switch may be any kind of suitable switch, e.g. a MOSFET such as a Si MOSFET, a GaN MOSFET, or a Sic MOSFET, or a bipolar transistor. An LED group may e.g. comprise one or more LED(s), arranged in series and/or parallel to each other. The plurality of LED groups, or also defined as multi-group LEDs, are arranged in a parallel connection. For example, the multi-group LEDs may comprise a first LED group including one red LED, a second LED group including one blue LED and one green LED and a third LED group including one white LED. The different LED groups may e.g. be adapted to emit light with the same or different colour, temperature and/or intensity. Further, the LED fixture comprises a capacitor arranged in a parallel connection to the plurality of LED groups. Such a capacitor is applied in order to reduce a high frequency which may occur on the current supplied to the LED groups otherwise.

The LED driver is adapted to be powered by a single main power source. The LED driver comprises a power converter, e.g. an AC/DC converter, adapted to convert an input power provided by the power source to a current which is provided to the plurality of LED groups. The power converter may e.g. comprise a buck/boost converter, a flyback converter, a power factor corrector (PFC) flyback converter, or another PFC-converter such as a PFC boost converter or an LLC converter.

A control unit is configured to control the power converter, as such the power converter provides current to plurality of LED groups. The control unit may comprise any type of control unit, including e.g. analogue control electronics, digital control electronics, such as a micro controller, microprocessor, or any other suitable control device such as a Field Programmable Gate Array (FPGA), a programmable logic device (PLD), discrete logic electronics etc.

The current is provided alternately between the plurality of LED groups. Hence, during operation only one LED group is powered on, while the other LED groups are shut down. The control unit is configured to control the switch of each LED group for switching between the different LED groups. The control unit can e.g. be adapted to control the switch of each LED group by providing a voltage or current to the switch, e.g, wherein a voltage or current above a predetermined threshold results in the switch being on or off. When a current is supplied to a particular LED group, a forward voltage across the LED group is generated. Generally, the forward voltage associated with a LED group is different compared to other LED groups. Thus, each LED group has its unique forward voltage. Resuming the above example, wherein the multi-group LEDs comprise three LED groups. The first LED group including one red LED has the lowest forward voltage of the LED groups. The second LED group including one blue LED and one green LED has the highest forward voltage. The forward voltage of the third LED group is between the forward voltage of the other two LED groups.

When the power converter provides a current to the LED fixture, the LEDs of the LED group, e.g. a first LED group, which is switched on by the control unit light up and the capacitor arranged in parallel connection to the LED groups is being charged. The charging of the capacitor imposes a capacitor voltage across the capacitor. After providing the current to the first LED group, the first LED group is switched off and a second LED group is switched on by the control unit. Suppose that the first LED group has a higher forward voltage relative to the forward voltage of the second LED group, an extra current will flow through the second LED group when the switching occurs. The extra current originates from the discharging of the capacitor. This extra current induces a peak current through the second LED group, which could be detrimental to the lifetime or operating characteristics of the LEDs of the second LED group. Applied to the earlier example, switching from the second or third LED group to the first LED group leads to an unwanted peak current through the red LED. Hence, the first LED group is sensitive to the peak current.

The control unit of the LED driver according to the invention is configured to control the operation mode of the switch of the LED group between a nominal mode and a transition mode. In the nominal mode, the switch operates in a closed nominal state or an open nominal state. Typically, in the nominal mode the applied (nominal) current can continuously flow through an LED and which causes the LED to operate at a desired operating temperature or within a certain temperature range, so as to ensure a certain desired lifetime of the LED, e.g. expressed in illumination hours. In the closed nominal state, the switch associated to the LED group is closed such that the nominal current flows through the LED group. The LED group is thus switched on. In the open nominal state, the switch of the LED group is open such that no (nominal) current flows through the LED group. The LED group is thus switched off. In the transition mode, the switch operates in a semi-closed state, wherein the gate to source voltage of the switch of the LED group is lower than the gate to source voltage of the respective switch when operated in the closed nominal mode. For example, the nominal value of the gate to source voltage in the closed nominal mode may be set at 5 V, while the gate to source voltage may be decreased to e.g. 1.5 V when the switch operates in the semi-closed state.

Within the meaning of the present invention, gate to source voltage of a switch may also be referred to as the gate-source voltage or V_{GS} of the switch.

The control unit is further configured to switch the current from flowing through a first LED group to flowing through a second LED group, wherein the first LED group has a higher forward voltage than the second LED group, by switching the switch of the first LED group in the open nominal state and the switch of the second LED group in the semi-closed state during a transient period. The transient period is for example between 1-10 microseconds, preferably between 1-5 microseconds, more preferably between 1-3 microseconds. During this transient period, the switch of the second LED group temporary behaves like a resistor with a finite electrical resistance. This ensures that the flow of current through the second LED group is limited during the transient period. Consequently, the peak current is mitigated, or at least reduced, when switching from the first LED group to the second LED group.

After the transient period, the control unit controls the switch of the second LED group by switching the switch from the semi-closed state to the closed nominal state. The changing of the operation mode of the switch can be accomplished by increasing the gate to source voltage of the switch of the second LED group to the nominal value when switching from the semi-closed state to the closed nominal state.

In an embodiment, the LED driver according to the invention further comprises a first resistor. The first resistor comprises an input connectable to the control unit and an output connectable to the gate of the switch of the second LED group to arrange the first resistor in a serial connection with the gate of the switch of the second LED group. When the control unit is connected to the input of the first resistor, the control unit may transmit a control signal to the first resistor. The control signal may e.g. be represent a control voltage which is applied to the input of the first resistor. The control voltage may e.g. be 5V. When the output of the first resistor is connected to the gate of the switch of the second LED group, the control unit transmits the control signal to the input of the first resistor. As a result, a voltage is applied across the first resistor, resulting in the gate to source voltage of the switch to be lower than the applied control voltage of 5 V. Because the gate to source voltage is thus lower than the nominal gate to source voltage, e.g. 5 V, for operating in the closed nominal mode, the switch of the second LED group will operate in the transition mode by the control unit. The switch of the second LED group remains in the transition mode during the transient period. During the transient period, the voltage at the output of the first resistor builds up due to the flow of current through the first resistor, such that the gate to source voltage of the switch of the second LED group steadily increases towards the nominal value, e.g. 5 V. After the transient period, the gate to source voltage of the switch reaches the nominal value, wherein the switch of the second LED group is switched from the semi-closed state to the nominal closed state. The combination of the first resistor and the gate capacitance of the switch will cause a 1^{st} order RC response to the application of a control signal from the control unit. As such, when a control signal, e.g. 5V is applied to the input of the first resistor, the switch will go through a transient period before reaching the nominal closed state. When applying a typical value of 100 pF for the gate capacitance and a value of 10 kΩ for the first resistor, it can be shown that this will result in a transient period when a switch is switched from an off state or open state to an on state or closed state. During said transient period, the switch thus acts as a resistor rather than as a short circuit, thus limiting the current through the second LED group. Based on e.g. datasheets of the applied switch, one can evaluate the resistance value of the switch, i.e. the R_{DS} resistance, as a function of the gate to source voltage V_{GS} of the switch.

In an embodiment of the present invention, the control signal from the control unit as applied to the first resistor can be a pulsed signal, e.g. a PWM signal, rather than a continuous signal. In such embodiment, the control signal may comprise a plurality of pulses during a transient period, e.g. a transient period of a few usec. The pulsed signal as applied enables the application of a controlled Gate to Source voltage for the switch, thus controlling the resistance value of the switch, and thus controlling the current through the switch. In such embodiment, control of the current through the switch can be reached by controlling the frequency of the pulsed signal and/or the duty cycle of the pulsed signal.

In an embodiment, in order to further control the transient period, the control unit may be configured to adjust the control voltage, e.g. apply a reduced control voltage at the start of the transient period.

In a further embodiment, the LED driver according to the invention further comprises a first resistor switch which is arranged in a parallel connection with the first resistor. The first resistor switch may e.g. be a MOSFET. The control unit is configured to control the first resistor switch by switching the first resistor switch on, i.e. the first resistor switch is closed, or off, i.e. the first resistor switch is open. When the first resistor switch is switched open by the control unit, the first resistor is arranged in a serial connection with the gate of the switch of the second LED group.

The first resistor switch associated with a particular LED group will be switched open by the control unit, when the switched on LED group has a lower forward voltage than another LED group which is switched off. On the contrary, the first resistor switch associated with a particular LED group will be switched on by the control unit, when the switched on LED has a higher forward voltage than another LED group which is switched off. In the latter situation, no peak current problem arises due to the higher forward voltage, whereby it is not required to decrease the gate to source voltage temporary during the transient period. An advantage of using the first resistor switch is thus that the control unit is configured of controlling the gate to source voltage of the LED group that is switched on. In an embodiment, the control unit comprises a memory for storing configuration data of the plurality of LED groups of the LED fixture. The configuration data includes the (nominal) forward voltage of each LED group. The forward voltage data enables the control unit to decide whether the first resistor switch associated with the LED group that is switched on, needs to be switched on or off.

In another embodiment, the LED driver according to the invention comprises a second resistor. The second resistor is arrangeable in a serial connection with the LED fixture of the LED driver. When switching the current from flowing through a first LED group to flowing through a second LED group, wherein the first LED group has a higher forward voltage than the second LED group, the current also flows through the second resistor arranged in series with the LED fixture. This current will result in a voltage drop across the second resistor. Due to this additional voltage drop across the second resistor, the current through the second LED group will be limited.

In a further embodiment, the LED driver according to the invention further comprises a second resistor switch which is arranged in a parallel connection with the second resistor. The second resistor switch may e.g. be a MOSFET. The control unit is configured to control the second resistor switch. For example, the control unit may transmit a control signal to the second resistor switch, wherein the control signal results in the second resistor switch being on or off. When the second resistor switch is switched open by the control unit, the second resistor is arranged in a serial connection with the LED fixture of the LED driver.

In an embodiment, the control unit comprises a memory for storing configuration data of the plurality of LED groups of the LED fixture. The configuration data includes the (nominal) forward voltage of each LED group. The forward voltage data enables the control unit to decide whether the second resistor switch needs to be switched on or off based on the particular LED group that is switched on.

In an embodiment of the present invention, a LED system is provided, the LED system comprises the LED driver according to the present invention and a LED fixture comprising a plurality of LED groups. Each LED group comprises at least one LED forming a series connection with a switch, wherein the plurality of LED groups are arranged in a parallel connection. The LED fixture further comprises a capacitor arranged in a parallel connection to the plurality of LED groups. The LED fixture further comprises a resistor arranged in a serial connection with the plurality of LED groups and a resistor switch arranged in a parallel connection with the resistor. The control unit of the LED driver is configured to control the resistor switch, e.g. by transmitting a control signal to the resistor switch. Controlling the resistor switch allows to control the current through the LED group, due to the voltage drop across the resistor.

For example, the control unit may transmit a control signal to the resistor switch, wherein the control signal results in the resistor switch being on or off. When the resistor switch is switched open by the control unit, the resistor is arranged in a serial connection with the LED fixture. The open state of the resistor switch enables the resistor to operate the switch of the switched on LED group in the transition mode. After the transient period, the control unit switches the parallel resistor switch to a closed (nominal) state, e.g. by again transmitting a control signal to the parallel resistor switch. In the closed state of the parallel resistor switch, the switch of the switched on LED group operates in the nominal closed state.

The invention further relates to a method of controlling a LED fixture comprising a plurality of LED groups. Each LED group comprises at least one LED forming a series connection with a switch. The plurality of LED groups are arranged in a parallel connection. The LED fixture further comprises a capacitor arranged in a parallel connection to the plurality of LED groups. The method comprises the steps of:
controlling the operation mode of the switch of the LED group between a nominal mode, wherein the switch operates in a closed nominal state or an open nominal state, and
a transition mode, wherein the gate to source voltage of the switch of the LED group is lower than the gate to source voltage of the respective switch when operated in the closed nominal state,
switching the current from flowing through a first LED group to flowing through a second LED group, wherein the first LED group has a higher forward voltage than the second LED group, by switching the switch of the first LED group in the open nominal state and the switch of the second LED group in the transition mode during a transient period.

The invention will be described below with reference to the figures. These figures serve as examples to illustrate the invention, and will not be construed as limiting the scope of the claims. In the different figures, like features are indicated by the like reference numerals. In the figures:

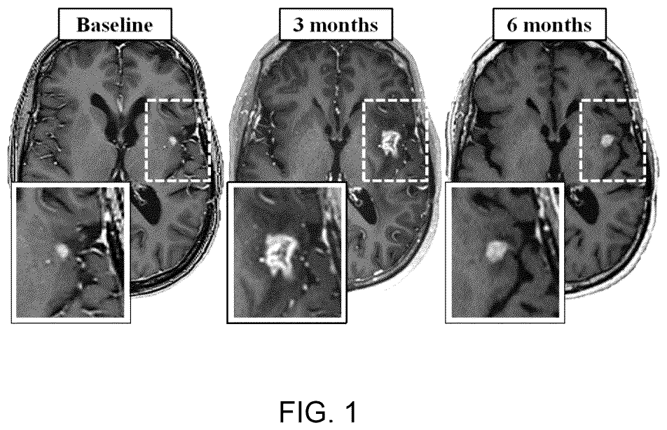

FIG. 1: Schematically illustrates a first embodiment of the invention;

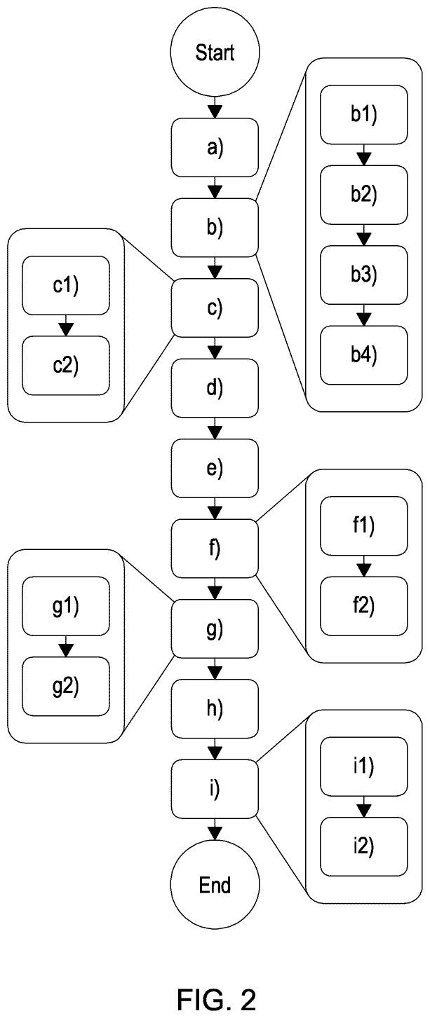

FIG. 2: Schematically illustrates a second embodiment of the invention;

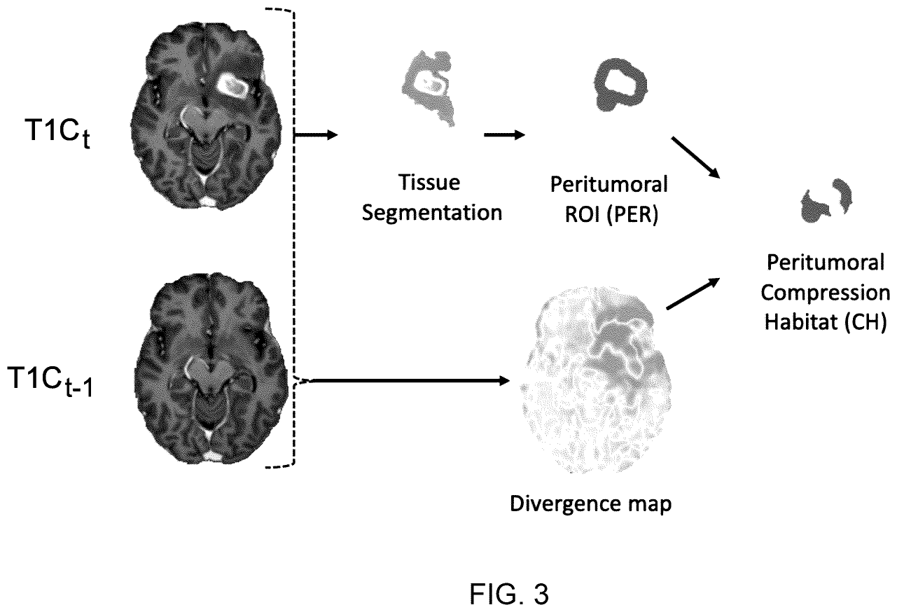

FIG. 3: Schematically illustrates an embodiment of a flow diagram of the method according the invention,

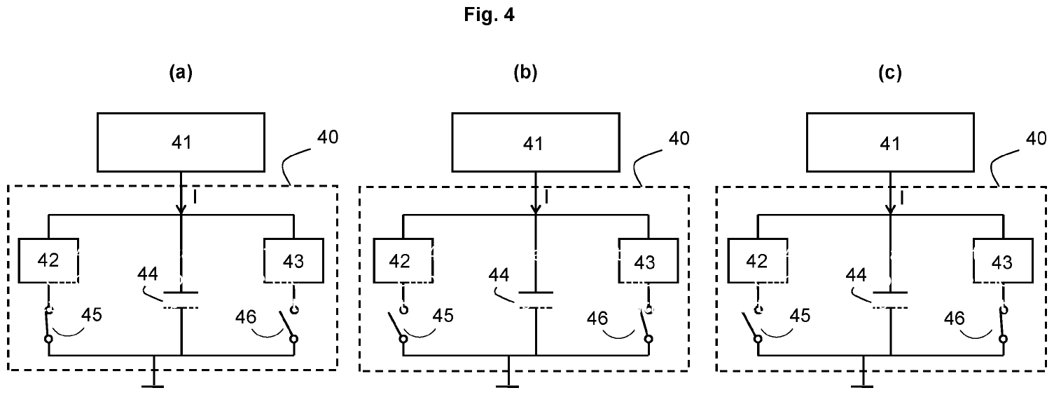

FIG. 4: schematically depicts an embodiment to illustrate the method according to the invention.

FIG. 1 schematically illustrates a first embodiment of the invention. The invention relates to a LED driver 1. The LED driver 1 controls a LED fixture 2 comprising a plurality of LED groups. The plurality of LED groups are arranged in a parallel connection. Each LED group may comprise any suitable number of LEDs arranged in series or parallel. In FIG. 1, the LED fixture 2 comprises three LED groups: a first LED group 2a comprising two LEDs, a second LED group 2b comprising a single LED and a third LED group 2c comprising a single LED. The LED(s) of each LED group form a series connection with a switch 3a, 3b, 3c. In the shown example, the switch 3a, 3b, 3c of each LED group is a MOSFET, but other types of switches can be used. The LED fixture 2 further comprises a capacitor 4 arranged in a parallel connection to the plurality of LED groups 2a, 2b, 2c.

The LED driver 1 is powered by a main power source 5, which in this case is a mains connection, e.g. supplying 230 V or 120V/277V at 50 Hz or 60 Hz. The LED driver 1 comprises a power converter 6, e.g. an AC/DC converter, adapted to convert an input power provided by the power source 5 to a (supply) current I which is provided to the plurality of LED groups to emit light. Alternatively, the LED driver may be configured to operate in a voltage mode and provide a supply voltage to the plurality of LEDs. The power converter 6 may e.g. comprise a buck/boost converter, a flyback converter, a power factor corrector (PFC) flyback converter, or another PFC-converter such as a PFC boost converter or a resonant converter such as an LLC or LCC converter.

The LED driver 1 further comprises a control unit 7. The control unit 7 is configured to control the power converter 6, as such the power converter 6 provides current to the plurality of LED groups 2a, 2b, 2c. The control unit 7 may comprise any type of control unit, including e.g. analogue control electronics, digital control electronics, such as a micro controller, microprocessor, or any other suitable control device such as a Field Programmable Gate Array (FPGA), a programmable logic device (PLD), discrete logic electronics etc.

The control unit 7 can as such control the plurality of LED groups 2a, 2b, 2c that the current is provided alternately between the plurality of LED groups 2a, 2b, 2c. Hence, during operation only one LED group is powered on, while the other LED groups are switched off. The control unit 7 is configured to control the switch 3a, 3b, 3c of each LED group for switching between the different LED groups 2a, 2b, 2c. The switching between the different LED groups may occur by means of applying suitable control signals 11 to the switches 3a, 3b, 3c. An LED group can be switched on when the control unit 7 is connected to a particular switch 3a, 3b, 3c of the LED group and is configured to control said switch. The control unit 7 is adapted to control the switch 3a, 3b, 3c of the LED group by providing a voltage or current to the switch 3a, 3b, 3c, e.g, wherein a voltage or current above a predetermined threshold results in the switch 3a, 3b, 3c being on or off. When a current is supplied to a switched on LED group 2a, 2b, 2c, a forward voltage across the LED group 2a, 2b, 2c is generated due to the current I supplied to the LED group.

Suppose that the first LED group 2a is switched on and the second 2b and third 2c LED group are switched off by the control unit 7. When the power converter 6 provides a current to the LED fixture 2, the LED of the first LED group 2a lights up. In addition, the capacitor 4 arranged in parallel connection to the LED groups 2a, 2b, 2c is being charged. The charging of the capacitor 4 imposes a capacitor voltage across the capacitor 4. After providing the current to the first LED group 2a, the first LED group 2a is switched off and the second LED group 2b is switched on by the control unit 7. Imagine that the first LED group 2a has a higher forward voltage relative to the forward voltage of the second LED group 2b, an extra current will flow through the second LED group 2b when switching to the second LED group 2b. The extra current originates from the discharging of the capacitor 4. This extra current induces a peak current through the second LED group 2b, which could be detrimental to the lifetime or operating characteristics of the LED of the second LED group 2b.

To mitigate the above problem, the LED driver 1 comprises a first resistor 8a, 8b, 8c for each LED group 2a, 2b, 2c. Each first resistor 8a, 8b, 8c comprises an input 9a, 9b, 9c and an output 10a, 10b, 10c. The input 9a, 9b, 9c of the first resistor is connectable to the control unit 7 and the output 10a, 10b, 10c is connectable to the gate of the switch 3a, 3b, 3c of the respective LED group to arrange the first resistor 8a, 8b, 8c in a serial connection with the gate of the switch 3a, 3b, 3c of the respective LED group. When the control unit 7 is connected to the input 9a, 9b, 9c of the first resistor 8a, 8b, 8c, the control unit 7 may transmit a control signal 11 to the first resistor 8a, 8b, 8c. The control signal 11 may e.g. be represent a control voltage which is applied to the input 9a, 9b, 9c of the first resistor. The control voltage may e.g. be 5 V. When the output 10a, 10b, 10c of the first resistor is connected to the gate of the switch 3a, 3b, 3c of the respective LED group, the control unit 7 transmits the control signal 11 to the input 9a, 9b, 9c of the first resistor. As a result, a voltage is applied across the first resistor 8a, 8b, 8c, wherein the gate to source voltage of the switch 3a, 3b, 3c decreases. Because the gate to source voltage is decreased, the switch 3a, 3b, 3c of the respective LED group is switched in a transition mode by the control unit 7. In the transition mode, the switch 3a, 3b, 3c operates in a semi-closed state, wherein the gate to source voltage of the switch 3a, 3b, 3c of the LED group is lower than the gate to source voltage of the respective switch when operated in a nominal mode. In the nominal mode, the switch 3a, 3b, 3c operates in a closed nominal state or an open nominal state.

The switch 3a, 3b, 3c of the respective LED group remains in the transition mode during a transient period. The transient period is for example between 1-10 microseconds, preferably between 1-5 microseconds, more preferably between 1-3 microseconds. During the transient period, the voltage at the output 10a, 10b, 10c of the first resistor builds up due to the flow of current through the first resistor 8a, 8b, 8c, such that the gate to source voltage of the switch 3a, 3b, 3c of the respective LED group steadily increases towards the nominal value, e.g. 5 V. After the transient period, the gate to source voltage of the switch 3a, 3b, 3c reaches the nominal value, wherein the switch 3a, 3b, 3c of the respective LED group is switched from the semi-closed state to the nominal closed state. In the shown example of FIG. 1, the control unit 7 switches the current from flowing through the first LED group 2a to flowing through the second LED group 2b, wherein the first LED group 2a has a higher forward voltage than the second LED group 2b. The control unit 7 switches simultaneously the switch 3a of the first LED group in the open nominal state and the switch 3b of the second LED group in the semi-closed state.

The LED driver 1 of FIG. 1 further comprises a first resistor switch 11a, 11b, 11c which is arranged in a parallel connection with the first resistor 8a, 8b, 8c. The first resistor switch 11a, 11b, 11c may e.g. be a MOSFET. The control unit 7 is configured to control the first resistor switch 11a, 11b, 11c by switching the first resistor switch 11a, 11b, 11c on, i.e. the first resistor switch 11a, 11b, 11c is closed, or off, i.e. the first resistor switch 11a, 11b, 11c is open. The control unit 7 can e.g. be adapted to control the first resistor switch 11a, 11b, 11c by providing a switch signal 12 representing a voltage or current to the first resistor switch 11a, 11b, 11c, e.g, wherein the voltage or current above a predetermined threshold results in the first resistor switch 11a, 11b, 11c being on or off. When the first resistor switch 11a, 11b, 11c is switched open by the control unit 7, the first resistor 8a, 8b, 8c is arranged in a serial connection with the gate of the switch 3a, 3b, 3c of the respective LED group.

The first resistor switch 11a, 11b, 11c associated with a particular LED group 2a, 2b, 2c will be switched open by the control unit 7, when the switched on LED group has a lower forward voltage than the LED group which is switched off. On the contrary, the first resistor switch 11a, 11b, 11c associated with a particular LED group 2a, 2b, 2c will be switched on by the control unit 7, when the switched on LED has a higher forward voltage than another LED group which is switched off. In the latter situation, no peak current problem arises due to the higher forward voltage, whereby it is not required to decrease the gate to source voltage temporary during the transient period.

In the shown example of FIG. 1, the control unit 7 may switch the current from flowing through the second LED group 2b to flowing through the first LED group 2a, wherein the first LED group 2a has a higher forward voltage than the second LED group 2b. The first resistor switch 11a associated with the first LED group 2a is switched on by the control unit 7 via the switch signal 12. Thereby, the first resistor 8a associated with the first LED group 2a is shorted.

An advantage of using the first resistor switch 11a, 11b, 11c is that the control unit 7 is configured of controlling the gate to source voltage of switch 3a, 3b, 3c of the LED group that is switched on. In an embodiment, the control unit 7 comprises a memory for storing configuration data of the plurality of LED groups 2a, 2b, 2c of the LED fixture 2. The configuration data includes the (nominal) forward voltage of each LED group. The forward voltage data enables the control unit 7 to decide whether the first resistor switch 11a, 11b, 11c associated with the LED group 2a, 2b, 2c that is switched on, needs to be switched on or off.

FIG. 2 schematically illustrates a second embodiment similar to FIG. 1 of the LED driver 1 according to the invention to drive the plurality of LED groups 2a, 2b, 2c of the LED fixture 2. Instead of the first resistor and/or the first resistor switch for each LED group, the LED driver 1 of FIG. 2 comprises a second resistor 20. The second resistor 20 is arrangeable in a serial connection with the LED fixture 2 of the LED driver via a second resistor switch 21. The second resistor switch 21 is arranged in a parallel connection with the second resistor 20. The second resistor switch 21 may e.g. be a MOSFET.

The control unit 7 is configured to switch the second resistor switch 21. For example, the control unit 7 transmits a control signal 22 to the second resistor switch 21, wherein the control signal 22 results in the second resistor switch 21 being on or off. When the second resistor switch 21 is switched open by the control unit 7, the second resistor 20 is arranged in a serial connection with the LED fixture 2 of the LED driver. When the control unit 7 switches the current from flowing through the first LED group 2a to flowing through the second LED group 2b, wherein the first LED group 2a has a higher forward voltage than the second LED group 2b, the current also flows through the second resistor 20 arranged in series with the LED fixture 2. This current results in a voltage drop across the second resistor 20. The voltage drop changes the gate to source voltage of the switch 3b of the second LED group 2b to a lower gate to source voltage value. Suppose that the provided current by the LED driver 1 is set at 1 A and the resistance value of the second resistor 20 is 2 Ohm. Due to the voltage drop across the second resistor 20, the gate to source voltage of the switch 3b will decrease with 2 V, e.g. from 5 V tot 3 V. The decrease of the gate to source voltage causes the switch 3b of the second LED group to operate in the transition mode. After the transient period, the control unit 7 switches the second resistor switch 21 to a closed (nominal) state, e.g. by again transmitting a control signal 22 to the second resistor switch 21. Thereby, the second resistor 20 is shorted. The gate to source voltage of the switch 3b of the second LED group increases back to the nominal value, e.g. 5 V, which means that the switch 3b operates in the nominal closed state. Controlling the second resistor switch 21 allows the control unit 7 to control the gate to source voltage of the switch 3a, 3b, 3c of the switched on LED group.

In an embodiment, the current I through the plurality of LED groups 2a, 2b, 2c as provided by the LED driver 1 can be determined from a current measurement circuit 23. The current measurement circuit 23 may comprise a resistance element, which resistance element is placed in serial connection with the LED fixture 2. The voltage across the resistance element combined with the know resistance value from the resistance element thus enables to determine the value of the current through the plurality of LED groups 2a, 2b, 2c, which current value could be fed back 24 to the control unit 7 by the current measurement circuit 23. In this embodiment, the current feedback loop 24 gives an extra check on the provided current by actively measuring the provided current.

In an embodiment, the control unit 7 comprises a memory for storing configuration data of the plurality of LED groups 2a, 2b, 2c of the LED fixture 2. The configuration data includes the (nominal) forward voltage of each LED group. The forward voltage data enables the control unit 7 to decide whether the second resistor switch 21 needs to be switched on or off based on the particular LED group 2a, 2b, 2c that is switched on.

FIG. 3 schematically depicts a flow diagram of an embodiment of the method according to the invention for controlling an LED fixture comprising a plurality of LED groups. Each LED group comprises at least one LED forming a series connection with a switch, wherein the plurality of LED groups are arranged in a parallel connection. The LED fixture further comprises a capacitor arranged in a parallel connection to the plurality of LED groups. The method according to the invention comprises a first step 30 of controlling the operation mode of the switch of the LED group between a nominal mode and a transition mode. In the nominal mode, the switch operates in a closed nominal state or an open nominal state. In the the transition mode, the gate to source voltage of the switch of the LED group is lower than the gate to source voltage of the respective switch when operated in the closed nominal state.

Further, the method comprises a second step 31, switching the current from flowing through a first LED group to flowing through a second LED group. The first LED group has a higher forward voltage than the second LED group. When the switching step 31 occurs, the switch of the first LED group is switched in the open nominal state and the switch of the second LED group is switched in the transition mode during a transient period. The transient period is for example between 1-10 microseconds, preferably between 1-5 microseconds, more preferably between 1-3 microseconds. During this transient period, the switch of the second LED group temporary behaves like a resistor with a finite electrical resistance. This ensures that the flow of current through the second LED group is limited during the transient period. Consequently, a peak current is mitigated, or at least reduced, when switching from the first LED group to the second LED group.

After the transient period, the switch of the second LED group may be switched from the semi-closed state to the closed nominal state. The changing of the operation mode of the switch can be accomplished by increasing the gate to source voltage of the switch of the second LED group to the nominal value when switching from the semi-closed state to the closed nominal state.

FIG. 4 schematically depicts an embodiment to illustrate the method according to the invention, wherein an LED fixture 40 comprising a plurality of LED groups is controlled by an LED driver 41. The LED driver 41 controls the LED fixture 40 by providing a current I. The LED driver 41 is also configured to provide a supply voltage to the LED fixture 40. In this embodiment, the LED fixture 40 comprises two LED groups arranged in a parallel connection: a first LED group 42 and a second LED group 43. Each LED group 42, 43 may comprise any suitable number of LEDs arranged in series or parallel. The LED fixture 40 further comprises a capacitor 44 arranged in a parallel connection to the two LED groups 42, 43. Each LED group 42, 43 forms a series connection with a switch, e.g. a MOSFET. The operation mode of the switch associated with LED group is controllable between a nominal mode and a transition mode. In the nominal mode, the switch operates in a closed nominal state or an open nominal state. In the transition mode, the gate to source voltage of the switch of the LED group is lower than the gate to source voltage of the respective switch when operated in the closed nominal state.

In situation (a), the switch 45 of the first LED group 42 is switched on and operates in the closed nominal mode. The switch 46 of the second LED group 43 is switched off and operates in the open nominal mode. The current I flows through the first LED group 42 and the capacitor 44. As a result, the first LED group 42 emits light and the capacitor is being charged.

In situation (b), the current I is switched from flowing through the first LED group 42 to flowing through the second LED group 43. The first LED group 42 has a higher forward voltage than the second LED group 43. As can be seen in situation (b) of FIG. 4, the switch 45 of the first LED group 42 is switched in the open nominal state and the switch 46 of the second LED group 43 is switched in the transition mode. The switch 46 of the second LED group 43 is not completely closed, i.e. semi-closed, during a transient period. During this transient period, the switch 46 of the second LED group 43 temporary behaves like a resistor with a finite electrical resistance. This ensures that the flow of current through the second LED group 43 is limited during the transient period. Consequently, a peak current is mitigated, or at least reduced, when switching from the first LED group 42 to the second LED group 43.

After the transient period, the switch 46 of the second LED group 43 is switched from the semi-closed state to the closed nominal state. This is visualised in situation (c) of FIG. 4. The changing of the operation mode of the switch can be accomplished by increasing the gate to source voltage of the switch 46 of the second LED group 43 to the nominal value when switching from the semi-closed state to the closed nominal state.

As required, detailed embodiments of the present invention have been described.

The various terms used in the description should not be interpreted as restrictive but rather as a comprehensive explanation of the invention.

The word “a” used herein means one or more than one, unless specified otherwise. The phrase “a plurality of” means two or more than two. The words “comprising” and “having” do not exclude the presence of more elements.

We claim:

1. A method of assessment of medical images of a brain including a lesion, the method comprising:
obtaining a stack of longitudinal medical images;
registering the stack of images to a common reference space by:
performing a rigid intrapatient registration of the longitudinal stack of images; and
performing an affine registration of the longitudinal stack of images to the reference space;
segmenting lesion tissues using automated methods and defining a region of interest based on dilation operations;
estimating the displacement field within each consecutive image pair within the stack of longitudinal medical images;
computing magnitude and divergence maps from the estimated displacement fields by:
computing the magnitude of the displacement field to characterise pixelwise displacement within the image field of view and dividing these pixelwise phenomena by the time between their associated consecutive image pair within the stack; and
computing the divergence of the displacement field to characterise pixelwise compression and/or expansion phenomena within the image field of view and dividing these pixelwise phenomena by the time between their associated consecutive image pair within the stack;
delineating the regions that display a divergence and thereby identifying a set of delineated regions; and
estimating displacement and/or divergence biomarkers by:
estimating the displacement degree in the delineated regions by computing a central tendency metric or a robust maximum estimator on magnitude map values distribution inside the region; and/or
estimating the divergence degree in the delineated regions by computing a central tendency metric or a robust maximum estimator on divergence map values distribution inside the region.

2. The method as claimed in claim 1, wherein the method comprises, after obtaining the stack of medical images, improving said stack of medical images prior to the step of registering the images by one or more of: subjecting each image of the stack to a noise filtering; eliminating from the images the areas corresponding to background or peripheral tissues of the region of interest; correcting inhomogeneities of the images; and/or normalizing intensities for morphological images.

3. The method as claimed in claim 1, wherein the step of registering the stack of images to a common reference space is done using linear transforms.

4. The method as claimed in claim 1, wherein the lesion tissues are tumour tissues and the method comprises segmenting tumour tissues using the automated methods in order to define a peritumoural region as the region of interest.

5. The method as claimed in claim 1, wherein the method includes estimating the displacement field within each consecutive image pair within the stack of longitudinal medical images.

6. The method as claimed in claim 5, wherein the estimation of displacement fields is done via use of a symmetric diffeomorphic image registration algorithm or of optical flow-based algorithms.

7. The method as claimed in claim 1, wherein the estimated displacement fields are subject to post-processing to remove spurious deformations due to registration errors and/or to remove noise or skull tissue not correctly removed.

8. The method as claimed in claim 7, wherein the lesion is a brain tumour and the post-processing is done though the steps of: removing spurious deformation based on tumour recurrence probability maps built on the presurgical location of the original tumour, and removing inconsistent deformations between continuous displacement fields.

9. The method as claimed in claim 1, wherein the step of delineating the regions that display a divergence involves regions with a positive divergence and the divergence biomarker is an expansion biomarker.

10. The method as claimed in claim 1, wherein the step of delineating the regions that display a divergence involves regions with a negative divergence and the divergence biomarker is a compression biomarker.

11. The method as claimed in claim 1, wherein the lesion is a brain tumour and the regions of interest are peritumoural regions, with the method comprising delineating the peritumoral region(s) that have a negative divergence to thereby delineate peritumoral regions affected by compression, or compression habitats.

12. The method as claimed in claim 1, wherein delineating the regions that display a divergence includes identifying regions where one or both of the computed divergence of the displacement field or the ratio of the computed divergence with time have a magnitude that exceeds a threshold value.

13. The method as claimed in claim 1, comprising providing a user, or some other computer system, with an output that includes the displacement biomarkers and/or the divergence biomarkers.

14. The method as claimed in claim 1, comprising display of output information in an image form depicting the computed magnitude and divergence maps and/or a map of the biomarkers.

15. The method as claimed in claim 1, wherein the stack of longitudinal medical images are images from an MRI imaging system.

16. (canceled)

17. (canceled)

18. A system for assessment of medical images of a brain including a lesion, the system comprising a data processing device configured to perform the method of claim 1.

19. The system as claimed in claim 18, being a computer system of an imaging system, wherein the imaging system is configured to obtain the stack of longitudinal medical images and provide them to the data processing device.

20. The system as claimed in claim 18, wherein the imaging system is an MRI imaging system.

21. (canceled)

22. A method of assessment of medical images of a brain including a lesion, the method comprising:
a) obtaining a stack of longitudinal medical images;
b) improving said stack of medical images, through the steps of:
b1. subjecting each image of the stack to a noise filtering;
b2. eliminating from the images the areas corresponding to background or peripheral tissues of the region of interest;
b3. correcting inhomogeneities of the images; and
b4. normalizing intensities for morphological images;
c) registering the improved stack of images obtained in step b) to a common reference space using linear transforms:
c1. performing a rigid intrapatient registration of the longitudinal stack of images;
c2. performing an affine registration of the longitudinal stack of images obtained in c1) to a reference space;
d) segmenting lesion tissues such as tumour tissues using automated methods such as a trained convolutional neural network and defining a region of interest, such as the peritumoural region, based on dilation operations;
e) estimating the displacement field within each consecutive image pair within the stack of longitudinal medical images previously obtained from c), using a non-linear registration algorithm such as the symmetric diffeomorphic image registration algorithm or optical flow-based algorithms;
f) post-processing the displacement fields obtained in e) to remove spurious deformations due to registration errors, noise or skull tissue not correctly removed, between others, though the steps of:
f1. removing spurious deformation based on tumour recurrence probability maps build on the presurgical location of the original tumour
f2. removing inconsistent deformations between continuous displacement fields
g) computing magnitude and divergence maps from displacement fields processed in f) by:
g1) computing the magnitude of the displacement field to characterise pixelwise displacement within the image field of view and divide it by the time between their associated consecutive image pair used in e);
g2) computing the divergence of the displacement field to characterise pixelwise compression or expansion phenomena within the image field of view and divide it by the time between their associated consecutive image pair used in e);
h) delineating the regions with a negative and/or positive divergence;
i) estimating displacement and/or compression/expansion biomarkers by:
i1. estimating the displacement degree in the region/habitat defined in h) by computing a central tendency metric or a robust maximum estimator on divergence values distribution inside the region; and/or
i2. estimating the compression/expansion degree in the region/habitat defined in h) by computing a central tendency metric or a robust maximum estimator on divergence values distribution inside the region.
